(19) United States
(12) Patent Application Publication
(10) Pub. No.: US 2010/0061950 A1
(43) Pub. Date: Mar. 11, 2010

(54) Antiperspirant/Deodorant Compositions and Methods

(75) Inventors: Vijay K. Joshi, Livingston, NJ (US); Philip Franco, Ocean Grove, NJ (US)

Correspondence Address: Joy S. Goudie;REVLON CONSUMER PRODUCTS CORPORATION, 237 PARK AVENUE, NEW YORK, NY, 10017, US

(73) Assignee: Revlon Consumer Products Corporation, New York, NY (US)

(21) Appl. No.: 12/593,019

(22) PCT Filed: Mar. 25, 2008
(86) PCT No.: PCT/US08/58139

§ 371 Date: Sep. 25, 2009

(30) Foreign Application Priority Data
Mar. 27, 2007 (US) 11691810

(51) Int. Cl. A61K 8/72; A61K 8/81; A61Q 15/00

(52) U.S. Cl. 424/65

(57) ABSTRACT

An aqueous based antiperspirant/deodorant composition comprising (a) an antiperspirant active ingredient; (b) at least one water absorbing polymer pretreated with a non-aqueous polar solvent; and (c) water, wherein the pretreated water absorbing polymer inhibits the water absorbing properties of the polymer in the aqueous based composition prior to application to the skin.

### TECHNICAL FIELD

The invention is in the field of antiperspirant/deodorant compositions, specifically those containing water.

Antiperspirants and deodorants function differently. An antiperspirant contains aluminum salts that are believed to act by constricting skin pores so that perspiration is reduced or eliminated. On the other hand, deodorants do not stop perspiration, but rather, contain an anti-bacterial ingredient that prevents bacterial growth and decomposition of perspiration, which is what causes perspiration malodor. While the aluminum salts used in antiperspirants are well known to be safe and efficacious, there are many reasons to try and formulate products that will reduce or eliminate underarm wetness and counteract perspiration malodor without using antiperspirant salts.

One way antiperspirant and deodorant manufacturers have tried to counteract the wetness problem is by formulating products with various types of water absorbing polymers, such as celluloses, sodium polyacrylates, and so on. Such water absorbing materials are well known for use in diapers. Such water absorbing materials absorb many times their weight in water and, when used in antiperspirants and deodorants, will greatly contribute to eradicating wetness. However, because such water absorbing materials are so effective at absorbing water, this makes it difficult to formulate them into compositions that contain water. For example, when a typical water absorbing polymer such as sodium polyacrylate is formulated into a standard aqueous based antiperspirant composition, the polymer tends to absorb the water in which the antiperspirant salt has been dissolved. This causes the antiperspirant salts dissolved in the aqueous portion of the composition to become much more concentrated. This, in turn, compromises the aesthetics of the antiperspirant such that when it is applied to skin it sometimes causes a gritty sensation.

For this reason, many antiperspirant manufacturers who use water absorbing materials use them in anhydrous compositions, or compositions that contain little or no water. However, this provides disadvantages as well. It is generally believed that aqueous based antiperspirants and deodorants provide better aesthetics. For example, when applied to the skin they provide a cooling sensation. The antiperspirant salts used can be dissolved in the aqueous portion of the composition so that they are not in the form of particulates, which can sometimes cause a gritty sensation when applied to skin.

Accordingly, there is a need for antiperspirant and deodorant formulations that contain high levels of water and at the same time a concentration of water absorbing polymer that is sufficient to provide a significant absorption of perspiration wetness. It has been found that aqueous based antiperspirant or deodorant compositions containing high levels of water absorbing materials can be formulated within certain parameters. These compositions provide excellent application properties and aesthetics, and at the same time are capable of absorbing considerable amounts of perspiration wetness.

It is an object of the invention to provide aqueous based antiperspirant and/or deodorant compositions containing a water absorbing material in an amount sufficient to enable the composition to absorb perspiration wetness.

It is a further object of the invention to provide aqueous based antiperspirant and/or deodorant compositions containing a water absorbing material in an amount sufficient to absorb perspiration wetness when the composition is applied to the skin.

It is a further object of the invention to provide a method for inhibiting perspiration wetness by treating the skin with an aqueous based composition containing a water absorbing material in an amount that is sufficient to absorb perspiration wetness on the skin.

### SUMMARY OF THE INVENTION

An aqueous based antiperspirant/deodorant composition comprising at least one water absorbing polymer pretreated with a non-aqueous polar solvent in an amount sufficient to inhibit the water absorbing properties of the polymer in the aqueous based composition prior to application to skin.

Another aspect of the present invention is an aqueous based antiperspirant/deodorant composition comprising (a) an antiperspirant active ingredient, (b) at least one water absorbing polymer pretreated with a non-aqueous polar solvent, and (c) water, wherein the pretreated water absorbing polymer inhibits the water absorbing properties of the polymer in the aqueous based composition prior to application to skin.

The present invention also includes a method for preparing an aqueous based antiperspirant/deodorant composition containing at least one water absorbing polymer in an amount sufficient to absorb perspiration wetness comprising pretreating a water absorbing polymer with a non-aqueous polar solvent prior to incorporation into the aqueous based composition.

Yet another embodiment of the invention is a method for improving an antiperspirant/deodorant composition comprising the step of pretreating a water absorbing polymer with a non-aqueous polar solvent prior to incorporation into the antiperspirant/deodorant composition.

### DETAILED DESCRIPTION

### I. The Composition

The invention is directed to an antiperspirant/deodorant composition containing at least one water absorbing polymer, e.g., a superabsorbent polymer, that is pretreated with a non-aqueous polar solvent in an amount sufficient to inhibit the water absorbing properties of the polymer while it is in the antiperspirant/deodorant composition. However, once the composition is applied to the skin the water absorbing polymer will be sufficiently operable to absorb perspiration wetness.

The aqueous based antiperspirant/deodorant composition comprises (a) an antiperspirant active ingredient, (b) at least one water absorbing polymer pretreated with a non-aqueous polar solvent, and (c) water. The pretreatment of the water absorbing polymer inhibits the polymer's ability to absorb water prior to application to the skin.

The composition includes water, which is added in an amount from about 0.5 to about 95% by weight of the total composition. Preferably, from about 5 to about 90%, and more preferably from about 10 to about 80% by weight of the total composition is added water.

Ingredients such as, for example, oils, esters, ethers, siloxane fluids, or hydrocarbons may also be included in the composition.

### A. The Water Absorbing Polymer

A variety of water absorbing polymers may be used in the compositions of the invention so long as they are capable of pretreatment with a non-aqueous polar solvent that will inhibit the water absorbing properties of the polymer while it is in the aqueous based composition, and when the composition is applied to the skin the water absorbing polymer will be effective to absorb perspiration wetness. The water absorbing polymers may be water soluble or water insoluble. In the latter case, the water insolubility is typically conferred by internal crosslinking of the polymeric matrix.

Typically, the water absorbing polymer is capable of absorbing at least about 0.1 grams of water per gram of material. Preferably, the water absorbing polymer is capable of absorbing about 0.05 grams to about 15 grams of water per gram of material, more preferably, about 0.1 grams to about 10 grams of water per gram of material, and most preferably, about 0.1 grams to about 3 grams of water per gram of material.

The water absorbing polymer is included in the composition, specifically from about 0.1 to about 80%, preferably from about 0.1 to about 70%, more preferably from about 0.1 to about 30%, and even more preferably from about 0.1 to about 5% by weight of the total composition.

The various types of water absorbent polymers include, but are not limited to those set forth herein.

### 1. Sodium Polyacrylates

Suitable sodium polyacrylates are polymers of acrylic acid, methacrylic acid, or their simple esters, such as C₁₋₄ esters, having a high content of COO— and Na+ ions. The sodium polyacrylates may be crosslinked or uncrosslinked. Crosslinking of the polymer will tend to make it water insoluble. On the other hand, if the polymer is not crosslinked or minimally crosslinked, it will tend to be more water soluble.

In the most preferred embodiment of the invention, the sodium polyacrylate used is a crosslinked water insoluble sodium polyacrylate. Most suitable are sodium polyacrylates sold under the brand name Aqua-Keep® by Kobo Products, Inc. including Aqua-Keep® J-440, SAGON Type II, J-550, or 10SH-NF. Particularly preferred is sodium polyacrylate sold by Kobo Products under the brand name Aqua-Keep® 10SH-NF, which has an absorption capacity of about 60 grams per gram of material, at an absorption rate of about 8 sec., a bulk density of about 0.87 g/ml, with a particle size of under 100 microns, or ranging from about 25 to about 100 microns, more preferably from about 50 to about 100 microns.

### 2. Polyacrylates

Also suitable as the water absorbing polymer are various types of polyacrylates sold under the Water Lock® trademark, such as Water Lock® C-200, Water Lock® G-400, Water Lock® A-240, Water Lock® A-220, Water Lock® A-180, Water Lock® A-120, Water Lock® A-100.

Water Lock® C-200 is a light tan, free flowing granular powder that absorbs or immobilizes large quantities of aqueous fluids at neutral or alkaline pH, having the INCI name corn starch/acrylamide/sodium acrylate copolymer or starch/acrylates/acrylamide copolymer. It has the following specifications: Absorbency: distilled water (ml/g)—300 min., aqueous 1% NaCl (ml/g)—60 min. pH 7.0 to 8.5. Sieve Test: % through U.S. #20-98.0 min.

Water Lock® G-400 is a light tan, free flowing granular powder that absorbs aqueous fluids at neutral or alkaline pH, having the INCI name acrylamide/sodium acrylate copolymer. It has the following specifications: Absorbency: distilled water (ml/g)—600 min., aqueous 1% NaCl (ml/g)—75 min. pH 7.0 to 8.5. Sieve Test: % through U.S. #20-95.0 min.

Water Lock® A-240 is a light tan free flowing granular powder that absorbs aqueous fluids at neutral or alkaline pH, having the INCI name corn starch/acrylamide/sodium acrylate copolymer or starch/acrylates/acrylamide copolymer. It has the following specifications: Absorbency: distilled water (ml/g)—260 min., aqueous 1% NaCl (ml/g) 40 min. pH 7.0 to 8.5. Sieve Test: % through U.S. #80-98.0 min.

Water Lock® A-220 is a free flowing fine granular powder that absorbs fluids at neutral or alkaline pH, having the INCI name corn starch/acrylamide/sodium acrylate copolymer or starch/acrylates/acrylamide copolymer. It has the following specifications: Absorbency: distilled water (ml/g)—300 min., aqueous 1% NaCl (ml/g)—40 min. pH 7.0 to 8.5. Sieve Test: % through U.S. #40-98.0 min.

Water Lock® A-180 is a light tan free flowing powder that absorbs aqueous fluids at neutral or alkaline pH having the INCI name corn starch/acrylamide/sodium acrylate copolymer or starch/acrylates/acrylamide copolymer. It has the following specifications: Absorbency: distilled water (ml/g)—120 min., aqueous 1% NaCl (ml/g)—30 min. pH 7.0 to 8.5. Sieve Test: % through U.S. #200-98.0 min.

Water Lock® A-120 is a light tan free flowing powder that absorbs aqueous fluids at neutral or alkaline pH, having the INCI name corn starch/acrylamide/sodium acrylate copolymer or starch/acrylates/acrylamide copolymer. It has the following specifications: Absorbency: distilled water (ml/g)—130 min., aqueous 1% NaCl (ml/g)—30 min. pH 7.0 to 8.5. Sieve Test: % through U.S. #40-98.0 min.

Water Lock® A-100 is a light tan, free flowing powder that can absorb large quantities of aqueous fluids at neutral or alkaline pH, having the WO name corn starch/acrylamide/sodium acrylate copolymer or starch/acrylates/acrylamide copolymer. It has the following specifications: Absorbency: distilled water (ml/g)—130 min., aqueous 1% NaCl (ml/g)—30 min. pH 7.0 to 8.5. Sieve Test: % through U.S. #20-98.0 min.

### B. The Non-Aqueous Polar Solvent

In order to ensure that the water absorbing polymer will not absorb the water in the antiperspirant/deodorant composition when it is in the resting state prior to application to skin, it is pretreated with a non-aqueous polar solvent prior to incorporation into the composition. The pretreatment procedure is accomplished by simply mixing the water absorbing polymer and non-aqueous polar solvent together using any suitable means. For example, the water absorbing polymer and the non-aqueous polar solvent may be combined using a propeller mixer. In another embodiment, the pretreatment procedure is accomplished by adding the water absorbing polymer and the non-aqueous polar solvent into a kettle and stirring the mixture with a paddle.

The non-aqueous polar solvent is included in the composition, in an amount sufficient to inhibit the water absorbing properties of the polymer in the aqueous based composition prior to application to the skin. The ratio of the water absorbing polymer to non-aqueous polar solvent is about 1:0.1 to about 1:15, preferably about 1:0.1 to about 1:10, more preferably about 1:0.5 to about 1:10, and even more preferably about 1:1 to about 1:10.

Suitable non-aqueous polar solvents include mono-, di-, or polyhydric alcohols, or more specifically solvents referred to as polyols or glycols.

### 1. Polyols

Suitable polyols are defined as compounds that contain three or more hydroxyl groups per molecule. Examples of suitable polyols include fructose, glucamine, glucose, glucose glutamate, glucuronic acid, glycerin, 1,2,6-hexanetriol, hydroxystearyl methylglucamine, inositol, lactose, malitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, dipropylene glycol, propylene glycol, butylene glycol, pentylene glycol, xylitol, or mixtures thereof.

### 1 Ethers

Also suitable as a nonaqueous polar solvent are various types of homopolymeric or block copolymeric liquid ethers. Polymeric ethers are preferably formed by polymerization of monomeric alkylene oxides, generally ethylene or propylene oxides. Preferred monomeric ethers are those exhibiting the structure below where n=1. Preferred polymeric ethers are comprised of moieties having the general structure below where n=2 to 100:

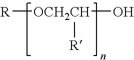

where R and R′ are each independently H, or C₁₋₃₀ straight or branched chain alkyl, and n is 1 to 20. Examples of such polymeric ethers include PEG, PPG, PEG/PPG copolymers, and derivatives thereof as well as alkoxylated alcohols such as steareth 2-100, ceteth 2-100, and the like.

Other examples of suitable polymeric ethers include polyoxypropylene polyoxyethylene block copolymers having the general formula:

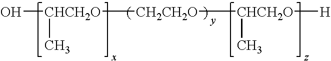

wherein x is 1-200, y is 1-200 and z is 1-200. Such compounds are sold under the CTFA names Meroxapol 105, 108, 171, 172, 174, 178, 251, 252, 254, 255, 258, 311, 312, and 314.

### 3. Alcohols

Mono- and dihydric alcohols are also suitable for use as the nonaqueous polar solvent. Generally, these mono- and dihydric alcohols have the general formula R(OH)ₙ where n is 1 or 2 and R is a substituted or unsubstituted saturated C₂₋₁₀, preferably C₁₋₈ alkyl, or a substituted or unsubstituted alicyclic, bicyclic, or aromatic ring, with the substituents selected from halogen, alkoxy, hydroxy, and so on. Examples of suitable alcohols include monohydric alcohols such as ethanol, isopropanol, hexyldecanol, benzyl alcohol, propyl alcohol, and isopropyl alcohol, as well as dihydric alcohols such as hexylene glycol, diethylene glycol, ethylene glycol, propylene glycol, 1,2-butylene glycol, triethylene glycol, dipropylene glycol, methyl propanediol, and mixtures thereof.

### 4. Sorbitan Derivatives

Sorbitan derivatives, which are defined as ethers or esters of sorbitan, are also suitable gel structure modifiers. Examples of suitable sorbitan derivatives are the Polysorbates, which are defined as stearate esters of sorbitol and sorbitan anhydrides, such as Polysorbate 20, 21, 40, 60, 61, 65, 80, 81, and 85. Also suitable are fatty esters of hexitol anhydrides derived from sorbitol, such as sorbitan trioleate, sorbitan tristearate, sorbitan sesquistearate, sorbitan stearate, sorbitan palmitate, sorbitan oleate, and mixtures thereof.

Preferred nonaqueous polar solvents include glycerin, propylene glycol, butylene glycol, pentylene glycol, and the like. Most preferred is where the water absorbing polymer is pre-treated with dipropylene glycol or glycerin prior to incorporation into the composition.

The compositions of the invention contain about 10.0%, preferably about 2.0%, more preferably about 1.0% by weight of the total composition of the water absorbing polymer pretreated with a non-aqueous polar solvent.

### C. Thickening Agents

The compositions of the invention preferably contain one or more thickening agents which may be water soluble or water insoluble. If present, the thickening agents range from about 0.1 to about 55%, preferably from about 0.5 to about 45%, more preferably from about 1 to about 40% by weight of the total composition.

### 1. Water Soluble Thickening Agents

Various types of water soluble thickening agents may be present. Such water soluble thickening agents will generally thicken the aqueous phase of the composition. Examples of such thickening agents include carbohydrates such as cellulose, polysaccharides, and the like.

### (a). Polysaccharides

Various polysaccharides, also referred to as polysaccharide gallants, may be suitable. Suitable polysaccharides are water soluble and contain at least one saccharide moiety (e.g., a polyhydroxy aldehyde or ketone, or acid hydrolysis product thereof, which, preferably, has the general formula Cₓ(H₂O)_{y}). Examples of saccharide moieties include the D and L forms of glucose, fructose, xylose, arabinose, fucose, galactose, pyruvic acid, succinic acid, acetic acid, galactose, 3,6-anhydro-galactose sulfate, galactose-4-sulfate, galactose-2-sulfate, galactose-2,6-disulfate, mannose, glucuronic acid, mannuronic acid, guluronic acid, galactouronic acid, rhamnose, and so on. Preferably the polysaccharide has a molecular weight ranging from about 500 to about 15,000,000 daltons, more preferably about 5,000 to about 1,000,000, and even more preferably about 25,000 to about 500,000 daltons. Polysaccharide gallants, which fulfill the above criteria include polysaccharides such as galactans, galactomannans, glucomannans, polyuronic acids, and the like. Suitable galactans are agar, agarose, and kappa carageenan, iota carageenan, and lambda carageenan. Examples of suitable galactomannans are locust bean gum and guar; examples of glucans are cellulose and derivatives thereof, starch and derivatives, dextrans, pullulan, beta 1,3-glucans, chitin, xanthan, tamarind and the like; examples of glucomannans are konjac; examples of polyuronic acids are algin, alginates, pectins; examples of heteropolysaccharides are gellan, welan, gum arabic, karaya gum, okra gum, aloe gum, gum tragacanth, gum ghatti quinceseed gum, psyllium, starch arabinogalactan and so on.

Preferred are galactans, in particular agarose, which is a polysaccharide comprised of basic repeating units of 1,3-linked beta-D-galactopyranose and 1,4-linked 3,6-anhydro-alpha-L-galactopyranose saccharide moieties. The agarose may be substituted by hydrophobic or hydrophilic groups. Examples of hydrophobic groups are alkoxy, in particular, methoxy. Examples of hydrophilic or polar groups are sulfate, pyruvate and the like. Examples of such substitutions are taught in Aoki, T. T.; Araki & M. Kitamikado; 1990, Vibrio sp. AP-2. Eur. J. Biochem, 187, 461-465, which is hereby incorporated by reference. The average molecular weight of agarose ranges between 35,700 and 144,000 daltons. The agarose suitable for use in the compositions of the invention may be from any suitable source or locale. For example an article authored by M. Lahaye and C. Rochas, Hydrobiologia, 221, 137-148, 1991, which is hereby incorporated by reference, discusses the numerous different types of agarose from different origins of seaweed species, all of which are suitable for use in the compositions of the invention. Also suitable for use in the compositions of the invention are chemically modified agaroses, such as those taught in an article authored by K. B. Guiseley in Industrial Polysaccharides: Genetic Engineering, Structure/Property Relations and Applications, Edited by M. Yalpani, 1987, Elsevier Science Publishers, which is hereby incorporated by reference. The Guiseley article teaches methods for the chemical modification of agaroses to obtain optimum gelling properties. One example of modified agarose is a hydroethylated agarose, which is sold under the brand names SeaPlaque and SeaPrep. In general, any modification of agarose that does not affect the helical conformation (i.e., which is obtained via linkage of the O6 and O4 of galactose to the O2 of 3,6-anhydrogalactose) will preserve the gelling capability.

In the most preferred embodiment of the invention, the composition contains at least two polysaccharide gellants, preferably a galactan and one gellant that is a galactomannan, glucan, glucomannan, polyuronic acid, or heteropolysaccharide. Most preferred is a composition comprising two gellants, agarose and locust bean gum in a range of about 0.05-10% by weight of the total composition of agarose, and 0.05-20% by weight of the total composition of locust bean gum. Agarose suitable for use in the compositions can be purchased from Seakem under the tradename Seakem LG agarose. Locust bean gum can be purchased from a number of sources including Centerchem or Pentapharm under the tradename Pentacare-HP.

### 2. Water Insoluble Thickening Agents

Various types of water insoluble thickening agents may be used in the composition of the invention, including but not limited to waxes, silicone elastomers, and the like.

### (a). Waxes

A variety of waxes may be used in the compositions of the invention including animal, vegetable, mineral, or silicone waxes. If present in the composition, the waxes may range from about 0.1 to about 50%, preferably about 0.5 to about 40%, more preferably about 1 to about 38% by weight of the total composition. Generally such waxes have a melting point ranging from about 28 to about 125° C., preferably about 30 to about 100° C. Examples of animal, vegetable, or mineral waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of ethylene homo- or copolymeric waxes such as polyethylene (also referred to as synthetic wax), polypropylene, and mixtures thereof.

Also suitable are various types of silicone waxes, referred to as alkyl silicones, which are polymers that comprise repeating dimethylsiloxy units in combination with one or more methyl-long chain (C₁₆₋₃₀) alkyl units where the long chain alkyl is preferably a fatty chain that provides a wax-like characteristic to the silicone. Such silicones include, but are not limited to stearoxydimethicone, behenoxy dimethicone, stearyl dimethicone, cetearyl dimethicone, cetyl dimethicone, and so on. Suitable waxes are set forth in U.S. Pat. No. 5,725,845, which is hereby incorporated by reference in its entirety.

### (b). Rheological Additives

The compositions of the invention may comprise one or more rheological additives. The term “rheological additive” means an ingredient or combination of ingredients that increase the viscosity of, or thicken, the composition, and if particulates are present, may also suspend the particulates in the composition.

### 1. Montmorillonite Mineral

One type of rheological additive comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, bentones, and the like. Another example of such a theological additive is silicate metal silicate gelling agents, such as those sold under the tradename Laponite®.

Also suitable as rheological additives are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonylphenyl

Another type of rheological additive that may be used in the compositions are silicas, silicates, silica silylate, and derivatives thereof. These silicas and silicates are generally found in the particulate form.

### D. Antiperspirant Active

The compositions of the invention contain about 1 to about 70%, preferably about 5 to about 70%, more preferably about 5 to about 25%, even more preferably from about 10 to about 22% by weight of the total composition of antiperspirant active salt.

The term “antiperspirant active salt” or “antiperspirant salt” means any compound or composition having antiperspirant activity, preferably astringent metallic salts such as the inorganic and organic salts of aluminum, zirconium, and zinc, and mixtures thereof. Particularly preferred are the aluminum and zirconium salts such as aluminum halides, aluminum hydroxide halides, zirconyl oxide halides, zirconyl hydroxy halides, and mixtures thereof. Aluminum salts include those of the formula:

Al₂(OH)ₐCl_{b}.xH2O

wherein a is from about 2 to 5; a+b=6; x is from about 1 to about 6; and wherein a, b, and x may have non-integer values. Zirconium salts include those of the formula:

ZrO(OH)₂₋ₐClₐ.xH₂O

wherein a is from about 1.5 to about 1.87; x is from about 1 to about 7; and wherein a and n may have non-integer values.

Examples of aluminum and zirconium salts include aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, and mixtures thereof.

Particularly preferred zirconium salts are those complexes also containing aluminum and glycine, in particular, aluminum zirconium tetrachlorohydrex GLY. The antiperspirant salts used in the composition of the invention are solubilized in the water. While preferably the antiperspirant salts are completely dissolved in the water, in some cases small amounts of salts may not be dissolved, i.e., may remain in the crystalline or suspensoid form.

### E. Silicones

The composition may also contain one or more silicones that may be liquid, solid or in the gum form. Also the silicones may be volatile or non-volatile. If present, the silicones may range from about 0.1 to about 80%, preferably from about 0.5 to about 75%, more preferably from about 1 to about 70% by weight of the total composition.

### 1. Volatile Silicones

Suitable volatile silicones are generally liquids at room temperature and include volatile linear or cyclic silicones. Generally such silicones have a viscosity ranging from about 0.1 to about 7 centistokes at 25° C. If present, suggested ranges of volatile silicone are from about 0.1 to about 80%, preferably about 0.5 to about 75%, more preferably about 1 to about 65% by weight of the total composition.

Cyclic silicones (or cyclomethicones) are of the general formula:

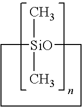

where n=3-6.

Linear volatile silicones that may be used in the compositions of the invention generally have the formula:

(CH₃)₃Si—O—[Si(CH₃)₂—O]ₙ—Si(CH₃)₃

where n=0-7, preferably 0-5, more preferably 1-4. Examples of such linear volatile silicones include hexamethyldisiloxane, octamethyltrisiloxane, decamethyltetrasiloxane, dodecamethylpentasiloxane, and mixtures thereof.

Linear and cyclic volatile silicones are available from various commercial sources including Dow Corning Corporation and General Electric. The Dow Corning volatile silicones are sold under the trade names Dow Corning 244, 245, 344, and 200 fluids. These fluids comprise octamethylcyclotetrasiloxane, decamethylcyclopentasiloxane, cyclohexasiloxane, and mixtures thereof.

### 2. Non-Volatile Silicones

Also suitable for use in the compositions of the invention are various non-volatile silicone oils, both water soluble and water insoluble. Such silicones preferably have a viscosity ranging from about 5 to about 499,000 centipoise, preferably about 10 to about 350,000 centipoise at 25° C. Suitable water insoluble silicones include amine functional silicones such as amodimethicone; phenyl substituted silicones such as phenyl trimethicone, phenyl dimethicone, trimethylsiloxyphenyl dimethicone, dimethicone, and the like. These types of silicone oils are available from a variety of sources including Dow Corning Corporation, GE Silicones, Wacker, and the like.

### F. Organic Oils

### 1. Hydrocarbons

The oil may comprise one or more volatile or non-volatile hydrocarbon oils. Examples of volatile hydrocarbons include various straight or branched chain paraffinic hydrocarbons having about 5 to about 40 carbon atoms, more preferably about 8 to about 16 carbon atoms. Suitable hydrocarbons include pentane, hexane, heptane, octane, decane, dodecane, tetradecane, tridecane, and C₈₋₂₀ isoparaffins such as isododecane, isohexadecane, and those disclosed in U.S. Pat. Nos. 3,439,088 and 3,818,105, both of which are hereby incorporated by reference. Preferred volatile paraffinic hydrocarbons have a molecular weight of about 70 to about 225, preferably about 160 to about 190 and a boiling point range of about 30 to about 320° C., preferably about 60 to about 260° C., and a viscosity of less than about 10 centipoise at 25° C. Such paraffinic hydrocarbons are available from EXXON under the ISOPARS trademark, and from the Permethyl Corporation.

Suitable nonvolatile hydrocarbon oils include longer chain isoparaffins and olefins, preferably those having greater than about 18 to about 20 carbon atoms. Examples of such hydrocarbon oils include C₂₄₋₂₈ olefins, C₃₀₋₄₅ olefins, C₂₀₋₄₀ isoparaffins; polyisobutene, polydecene, polybutene, and hydrogenated derivatives thereof; mineral oil, pentahydrosqualene, squalene, squalane, and mixtures thereof.

Also suitable are lower organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. Such silicones or organic oils include those further described as follows.

### 2. Esters

Suitable esters that may be used in the compositions of the invention are mono-, di-, and triesters. The composition may comprise one or more esters selected from the group, or mixtures thereof.

### (a). Monoesters

Monoesters are defined as esters formed by the reaction of a monocarboxylic acid having the formula R—COOH, wherein R is a straight or branched chain saturated or unsaturated alkyl having about 2 to about 50 carbon atoms, or phenyl; and an alcohol having the formula R—OH wherein R is a straight or branched chain saturated or unsaturated alkyl having about 2 to about 50 carbon atoms, or phenyl. Both the alcohol and the acid may be substituted with one or more hydroxyl groups, or may contain other groups such as ester, ether, and the like. Either one or both of the acid or alcohol may be a “fatty” acid or alcohol, and may have from about 6 to about 30 carbon atoms. Examples of monoester oils that may be used in the compositions of the invention include hexyldecyl benzoate, hexyl laurate, hexadecyl isostearate, hexyldecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

### (b). Diesters

Suitable diesters that may be used in the compositions of the invention are formed by the reaction of a dicarboxylic acid and an aliphatic or aromatic alcohol, or the reaction of an aliphatic or aromatic alcohol having at least two hydroxyl groups with one or more carboxylic acids. The dicarboxylic acid may contain from about 2 to about 50 carbon atoms, and may be in the straight or branched chain, saturated or unsaturated form. The dicarboxylic acid may be substituted with one or more hydroxyl groups. The aliphatic or aromatic alcohol may also contain about 2 to about 50 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e., contains about 14 to about 22 carbon atoms. The dicarboxylic acid may also be an alpha hydroxy acid. Examples of diester oils that may be used in the compositions of the invention include diisostearyl malate, esters of neopentyl glycol such as neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

### (c). Triesters

Suitable triesters comprise the reaction product of a tricarboxylic acid and an aliphatic or aromatic alcohol, or alternatively, the reaction of an aliphatic or aromatic alcohol having at least three hydroxyl groups with one or more carboxylic acids. As with the mono- and diesters mentioned above, the acid and alcohol contain about 2 to about 30 carbon atoms, and may be saturated or unsaturated, straight or branched chain, and may be substituted with one or more hydroxyl groups. Preferably, one or more of the acid or alcohol is a fatty acid or alcohol containing about 14 to about 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.

### (d). Tetraesters

Suitable tetraesters comprise the reaction product of alcohols having four hydroxyl groups such as pentaerythritol, with carboxylic acids which may be the same or different, and as described above with respect to the mono-, di-, and triesters. Examples of such tetraesters include esters of pentaerythritol and C₁₋₃₀ monocarboxylic acids. All of the hydroxyl groups may be reacted with monocarboxylic acids, or only one, two, or three.

### 3. Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

### 4. Fluorinated Oils

Also suitable for use in the composition are various fluorinated oils such as fluorinated silicones, fluorinated esters, or perfluoropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496, which is hereby incorporated by reference.

Perfluoropolyethers like those disclosed in U.S. Pat. Nos. 5,183,589; 4,803,067; and 5,183,588 (all of which are hereby incorporated by reference), are commercially available from Montefluos under the trademark Fomblin.

Fluoroguerbet esters are also suitable oils. The term “guerbet ester” means an ester that is formed by the reaction of a guerbet alcohol having the general formula:

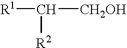

and a fluoroalcohol having the following general formula:

CF₃—(CF₂)ₙ—CH₂—CH₂—OH

wherein n is from 3 to 40.
with a carboxylic acid having the general formula:

R³COOH, or

HOOC—R³—COOH

wherein R¹, R², and R³ are each independently a straight or branched chain alkyl.

The guerbet ester may be a fluoro-guerbet ester, which is formed by the reaction of a guerbet alcohol and carboxylic acid (as defined above), and a fluoroalcohol having the following general formula:

CF₃—(CF₂)ₙ—CH₂—CH₂—OH

wherein n is from 3 to 40.

Examples of suitable fluoro guerbet esters are set forth in U.S. Pat. Nos. 5,488,121 and 5,312,968, which are hereby incorporated by reference.

The invention will be further described in connection with the following examples which are set forth for the purposes of illustration only.

### Example 1

An antiperspirant composition was made as follows:

### Phase A:

| Ingredient | % by weight |
|---|---|
| Water | 55.7 |
| Dipropylene glycol | 8.73 |
| Acetamide MEA | 2.92 |
| Agarose | 2.04 |
| Hydroxystearic acid | 17.5 |
| Pre-mix* | 13.12 |

*Pre-mix (% by weight):

| | |
|---|---|
| Cyclomethicone/dimethicone | 44.4 |
| crosspolymer | |
| PEG-12 dimethicone | 65.6 |
| Aluminum/zirconium | 64.0 |
| tetrachlorohydrex gly | |
| Phase A Mixture | 31.8 |
| Divinyldimethicone/dimethicone | 1.5 |
| crosspolymer, C12-13 pareth-23, | |
| C12-13 pareth-3** | |
| Zinc ricinoleate | 2.5 |
| Dipropylene glycol | 1.6 |
| Sodium polyacrylate | 0.1 |
| Fragrance | 1.0 |

**Tradename: HMW 2220 (Dow Corning)

Dipropylene glycol, acetamide MEA, and agarose were mixed in cold water and boiled at a temperature ranging from about 99° C. to about 102° C. until the mixture became clear. The mixture was cooled to 90° C. and hydroxystearic acid was added along with the pre-mix. The mixture was brought to and maintained at 75° C. Aluminum/zirconium tetrachlorohydrex gly, 64 grams, was added to 31.8 grams in Phase A, above. The temperature of the mixture was 58° C. 1.5 Grams of HMW 2220 (available from Dow Corning as a blend of divinyldimethicone/dimethicone crosspolymer, C12-13 pareth-23, and C12-13 pareth-3) was added to the mixture, followed by the addition of 2.5 grams of zinc ricinoleate in dipropylene glycol. Sodium polyacrylate, 0.1 gram, was wetted with 1 gram of dipropylene glycol and added to the mixture. The mixture was then poured into the appropriate containers and allowed to cool.

### Example 2

An antiperspirant composition was prepared as follows:

| Ingredient | % by weight |
|---|---|
| Water | 10.94 |
| Glycerin | 2.00 |
| Dipropylene glycol | 7.835 |
| Acetamide MEA | 1.00 |
| Agarose | 0.70 |
| Hydroxystearic acid | 6.00 |
| Pre-mix* | 4.50 |
| Aluminum/zirconium | 64.00 |
| tetrachlorohydrex glycine | |
| Sodium polyacrylate | 2.00 |
| Dipropylene glycol | 1.00 |
| Fragrance | 0.025 |

*Pre-mix:

| | |
|---|---|
| PEG-12 dimethicone copolyol | 55.55 |
| Dimethicone/vinyl dimethicone | 44.45 |
| crosspolymer | |

Water, glycerin, dipropylene glycol, acetamide MEA, and agarose were combined in a beaker and heated to 100° C. until a clear solution was formed. The mixture was then cooled to about 90° C. and allowed to stand for about 1 hour at room temperature. The temperature was maintained at about 78 to 80° C. The hydroxystearic acid and pre-mix were added and mixed at 73 to 75° C. Aluminum/zirconium tetrachlorohydrex glycine was preheated to 45° C., added to the mixture and the resulting mixture was brought to a temperature of about 58 to 60° C. Dipropylene glycol and fragrance were combined together and then added. The final mixture was poured into containers and allowed to cool at room temperature.

### Example 3

A deodorant composition was made as follows:

| Ingredient | % by weight |
|---|---|
| Water | 74.25 |
| Imidazolidinyl Urea | 0.20 |
| Citric Acid | 0.10 |
| Dipropylene Glycol | 12.00 |
| Zinc Ricinoleate | 0.25 |
| PPG-26 Buteth-26, PEG-40 | 3.00 |
| Hydrogenated Castor Oil | |
| Fragrance | 1.50 |
| Methylparaben | 0.20 |
| Sodium polyacrylate | 2.50 |
| Dipropylene Glycol | 3.00 |
| Water | 3.00 |

Water, imidazolidinyl urea, and citric acid were heated and mixed in kettle #1 at 40-45° C. Separately, in kettle #2, dipropylene glycol (12 grams), zinc ricinoleate, and methylparaben were combined, mixed and heated to 80-85° C. until uniform and completely dissolved. The mixture was then cooled to 70-75° C. while mixing. PPG-26 Buteth-26, PEG-40 Hydrogenated Castor Oil and fragrance were then added to kettle #2, which continued to mix until uniform. Dipropylene glycol (3 grams) and water were combined and mixed, then added to kettle #2 and further mixed for 15 to 20 minutes until the resulting mixture was free of lumps. Then the contents of kettle #2 were added to kettle #1. 3 Grams of water was used to flush kettle #2, which was then added to kettle #1, which was mixed using a sweep mixer until uniform. The resulting mixture was cooled to 20-35° C.

The product displayed the following properties:

Initial viscosity: 23-24 TD/5 rpm at 27° C.

Stabilized viscosity: 28.5 TD/5 rpm at 21° C.

Stabilized pH: 6.4 at 21° C.

### Example 4

A deodorant composition was made as follows:

| Ingredient | % by weight |
|---|---|
| Cyclopentasiloxane | 44.00 |
| Hydrogenated Polydecene | 24.00 |
| Stearamide MEA-stearate | 10.00 |
| Polyethylene | 2.00 |
| Pre-mix* | 10.00 |
| Sodium polyacrylate | 10.00 |

*Pre-mix (% by weight):

| | |
|---|---|
| PEG-12 dimethicone copolyol | 55.55 |
| Dimethicone/vinyl dimethicone | 44.45 |
| crosspolymer | |

Cylcopentasiloxane, hydrogenated polydecene, and stearamide MEA-stearate were added to a beaker, mixed and heated to 75° C. Sodium polyacrylate and the Pre-mix were combined and then added to the mixture. Mixing continued for about 15 minutes and the resulting mixture was then poured into the components.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. An aqueous based antiperspirant/deodorant composition comprising at least one water absorbing polymer pretreated with a non-aqueous polar solvent in an amount sufficient to inhibit the water absorbing properties of the polymer in the aqueous based composition prior to application to skin.

2. An aqueous based antiperspirant/deodorant composition comprising:
(a) an antiperspirant active ingredient;
(b) at least one water absorbing polymer pretreated with a non-aqueous polar solvent; and
(c) water,
wherein the pretreated water absorbing polymer inhibits the water absorbing properties of the polymer in the aqueous based composition prior to application to skin.

3. The composition of claim 1, wherein the water absorbing polymer has an absorption capacity of about 0.5 grams per gram of material.

4. The composition of claim 1, wherein the water absorbing polymer is crosslinked and water insoluble.

5. The composition of claim 1, wherein the water absorbing polymer is selected from the group consisting of acrylate/methacrylate.

6. The composition of claim 1, wherein the composition includes about 0.1 wt. % to about 30.0 wt. % of the water absorbing polymer.

7. The composition of claim 1, wherein the non-aqueous polar solvent is selected from the group consisting of polyols, ethers, alcohols, sorbitan derivatives and mixtures thereof.

8. The composition of claim 1, wherein the composition includes about 2.0 wt. % to about 70.0 wt. % of the non-aqueous polar solvent.

9. The composition of claim 1, wherein the composition includes about 10.0 wt. % to about 75.0 wt. % of the water.

10. The composition of claim 1, further comprising a thickening agent selected from the group consisting of water soluble thickening agents, water insoluble thickening agents, and mixtures thereof.

11. The composition of claim 1, further comprising a silicone compound selected from the group consisting of volatile silicone compounds, non-volatile silicone compounds, and mixtures thereof.

12. The composition of claim 1, further comprising an organic oil selected from the group consisting of hydrocarbon oils, esters, lanolin oils, fluorinated oils, and mixtures thereof.

13. The composition of claim 1, wherein the composition includes about 5.0 wt. % to about 70.0 wt. % of the antiperspirant active.

14. A method for preparing an aqueous based antiperspirant/deodorant composition containing at least one water absorbing polymer in an amount sufficient to absorb perspiration wetness comprising the step of pretreating a water absorbing polymer with a non-aqueous polar solvent prior to incorporation into the aqueous based composition.

15. A method for improving an antiperspirant/deodorant composition comprising the step of pretreating a water absorbing polymer with a non-aqueous polar solvent prior to incorporation into the antiperspirant/deodorant composition.

16. The method of claim 15, wherein the improvement is a reduction in tackiness or grittiness.

17. The method of claim 15, wherein the improvement is an increase in moisturizing.

18. The method of claim 15, wherein the improvement is an increase in efficacy.
